(19) United States
(12) Patent Application Publication
(10) Pub. No.: US 2011/0033058 A1
(43) Pub. Date: Feb. 10, 2011

(54) Display Apparatus

(75) Inventors: Takahiro KONDO, Osaka (JP); Hidenori Sato, Osaka (JP)

Correspondence Address: NDQ&M WATCHSTONE LLP, 300 NEW JERSEY AVENUE, NW, FIFTH FLOOR, WASHINGTON, DC, 20001, US

(73) Assignee: Sanyo Electric Co., Ltd., Osaka (JP)

(21) Appl. No.: 12/851,603

(22) Filed: Aug. 6, 2010

(30) Foreign Application Priority Data
Aug. 7, 2009 (JP) 2009-184357

(51) Int. Cl. A61F 11/06

(52) U.S. Cl. 381/71.3

(57) ABSTRACT

A display apparatus has a display unit; a housing which accommodates the display unit; a compressor for cooling the air inside the housing; a detecting unit which detects a sound generated from the compressor and an output unit which outputs a sound for canceling the sound detected by the compressor.

This application is based on Japanese Patent Application No. 2009-184357 filed on Aug. 7, 2009, the contents of which are hereby incorporated by reference.

### BACKGROUND OF THE INVENTION

1. Field of the Invention

The present invention relates to a display apparatus having a compressor for cooling an air inside the housing.

2. Description of Related Art

In recent years, display apparatuses called “digital signage” are emerging. Display apparatuses, which functions as an information board or an advertising pillar, are installed in many places, e.g. indoors and outdoors (JP2004-157499A).

The applicant have studied an display apparatus for installing outdoors, which equips cooling devices such as a compressor or an evaporator in order to control an internal temperature (for example, Japanese Patent Application Number 2008-145442). The cooling devices are equipped because the performance of a liquid crystal panel is degraded under a high temperature environment.

However, when such cooling devices are equipped inside the apparatus, a noise generates from the cooling device, where the noise interrupts person's voice or music. As a result, it makes audiences of the display apparatus difficult to capture the sound outputted from the display apparatus.

### SUMMARY OF THE INVENTION

A display apparatus of the present invention comprises a display unit; a housing which accommodates the display unit; a compressor for cooling the air inside the housing; a detecting unit which detects a sound generated from the compressor, and an output unit which outputs a sound for canceling the sound detected by the compressor.

The audio output unit may output a sound having an opposite phase to the sound detected by the detecting unit.

### BRIEF DESCRIPTION OF THE DRAWINGS

FIG. 1 shows a structure of a display apparatus. (a) is a front-view and (b) is cross-sectional view.

FIG. 2 is a functional block diagram of the display apparatus.

### DETAILED DESCRIPTION OF PREFERRED EMBODIMENTS

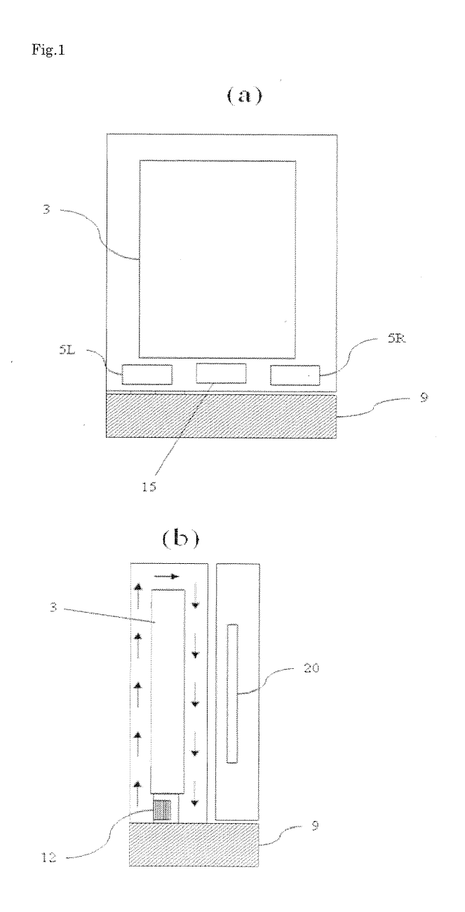

FIG. 1 shows an embodiment of the display apparatus of the present invention. (a) is its front-view, and (b) is the cross-sectional view.

The display apparatus has a display panel 3, a cooling device 9, a pair of speakers 5 (5L, 5R) which performs a stereo output, a microphone 12, a microphone 15, and an electronic circuit 20.

The display panel 3 displays an image and is constituted by a liquid crystal panel. When the display apparatus is installed outdoors, its temperature rises due to direct sunlight. Thus, in the present apparatus, an air cooled by the cooling device 9 is circulated in the perpendicular direction. In detail, the air cooled by the device 9 first flow the front side of the panel 3 from bottom up. Then the air flow the rear side of the panel 3 from top down. Then finally, the air is collected by the device 9. In this circulation path, a fan (not illustrated) for circulating the air is arranged.

The cooling device 9 is constituted by an evaporator, a compressor, and a condenser. The evaporator cools an air which came back to the cooling device 9 after circulating the circulation path. Then, the cooled air is emitted to the circulation path again. The compressor compresses a refrigerant flowing inside the evaporator. The condenser generates a liquefied refrigerant by cooling a refrigerant having a high temperature and high pressure, which was compressed by the compressor. The evaporator can cool the air in the cooling device 9 by running the liquefied refrigerant inside the evaporator.

Many of the power used in the cooling device 9 is used for the compressor which compresses the refrigerant. However, this compressor generates a loud noise. This causes a problem because the noise makes difficult for the audience to capture the sound outputted from display apparatus, or making people staying around the apparatus uncomfortable.

The electronic circuit 20 is constituted by an image output circuit, an audio output circuit, a system controller, etc. Further, the electronic circuit 20 analyzes the sound generated from the compressor, and performs a processing for generating the sound in order to cancel the noise from the compressor. In detail, the circuit 20 analyzes the frequency and the phase of an audio signal collected by the microphone 12, where the microphone 12 mainly collects the sound generated from the compressor. Then, the circuit 20 generates a signal whose phase is 180 degrees shifted from said audio signal, and then cancels the noise generated from the cooling device 9 by adding the audio signal and the phase-shifted signal. Further, the electronic circuit 20 is arranged outside the circulation path. The circuit 20 is arranged outside the circulation path because it can ease the maintenance and can shorten the circulation path.

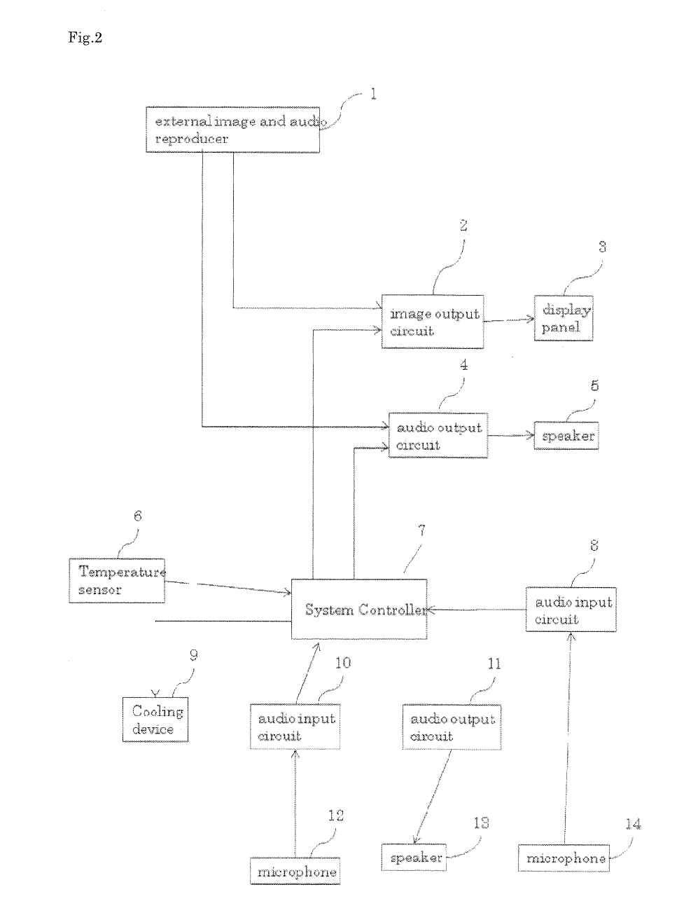

FIG. 2 is a functional block diagram of the display apparatus.

Regarding an image signal and an audio signal input from the external image-sound reproducer 1, the image signal is displayed on the display panel 3 via the image output circuit 2. The audio signal is outputted to the speaker 5 via the audio output circuit 4. The reproducer 1 may be a storage apparatus which stores an image contents, or the reproducer 1 may be a server connected by network with the present display apparatus.

Further, when temperature information acquired from the temperature sensor 6 is high, the system controller 7 operates the cooling device 9 and controls the temperature inside the display apparatus.

The microphone 12 collects a noise generated from the cooling device 9. The collected audio signal is transmitted to the system controller 7 via an audio input circuit 10. Here, the circuit 10 is intended for an input from the microphone 12. The system controller 7 analyzes the noise, then the controller 7 outputs an audio signal from the speaker 13, wherein the audio signal having a phase opposite to the noise, via audio output circuit 11. Here, the speaker 13 is intended for reducing the noise. The circuit 11 is intended for outputting an audio signal to the speaker 13. Thereby, the noise generated from the cooling device 9 is canceled and thus the noise is reduced.

Further, the microphone 14 collects an audio signal, in which the noise is reduced, and the collected signal is transmitted to the system controller 7 via the audio input circuit 8. Here the microphone 14 is intended for verifying whether the noise is reduced or not, and the circuit 8 is intended for inputting the audio signal from the microphone 14. When the noise is not sufficiently reduced, the noise collected by the microphone 14 and its anti-phase audio signal are added. The added signal is outputted from the speaker 13, and thus the noise is reduced.

The above-mentioned image output circuit 2, the audio output circuit 4, the system controller 7, the audio input circuit 8, the audio input circuit 10, and the audio output circuit 11 of FIG. 2 constitutes the electronic circuit 20 of FIG. 1.

A function of the microphone 14, which is shown in FIG. 2 and which is intended for verifying whether the noise is reduced, may be integrated to microphone 15 shown in FIG. 1. In this case, the system controller 7 analyzes the audio signal inputted from the audio input circuit 8, and determines whether the audio signal includes a noise component. If the noise component is included, the audio output signal 11 generates the other sound, which is also considered to be effective for reducing noise, from the speaker 13.

A function of the speaker 13, which is intended for reducing the noise, may be integrated to the speaker 5 shown in FIG. 1. In this case, an audio signal outputted from the audio output circuit 11, having an opposite phase to the noise, may be inputted to the audio output circuit 4 (instead of to the speaker 13), and may be composed with an audio signal inputted from the reproducer 1. Then, the composed signal may be outputted to the speaker 5.

A plurality of speakers 13 may be arranged near the compressor of the cooling device 9 so as to surround the compressor. Or instead, four small speakers may be arranged at the left, right, upper and lower position of the speaker 5, and these small speakers may function as a speaker 13 (a speaker for reducing noise).

When the temperature inside the display apparatus is low enough, it is not necessary to drive the cooling device 9. In this case, the microphone 12 and the speaker 13 may be turned off.

The embodiment of the present invention is described as above. However, the scope of the present invention is not limited thereto, and the present invention may be implemented by being subjected to various modifications without departing from the gist of the present invention.

What is claimed is:

1. A display apparatus comprising:
a display unit;
a housing which accommodates the display unit;
a compressor for cooling the air inside the housing;
a detecting unit which detects a sound generated from the compressor, and
an output unit which outputs a sound for canceling the sound detected by the compressor.

2. The apparatus of the claim 1, wherein
the audio output unit outputs a sound having an opposite phase to the sound detected by the detecting unit.
